(19) United States
(12) Patent Application Publication
(10) Pub. No.: US 2019/0009049 A1
(43) Pub. Date: Jan. 10, 2019

(54) Digital command prompting device for dementia patients using augmented reality

(71) Applicant: Katrina Goff Candy, Moama (AU)

(72) Inventor: Katrina Goff Candy, Moama (AU)

(21) Appl. No.: 15/999,813

(22) Filed: Aug. 23, 2018

Related U.S. Application Data
(63) Continuation-in-part of application No. 15/607,516, filed on May 28, 2017, now Pat. No. 9,922,169.

(51) Int. Cl. A61M 21/00; G08B 21/24; G06T 11/60; G10L 15/22

(52) U.S. Cl. CPC A61M 21/00; G08B 21/24; G06T 11/60; G10L 15/22; G10L 2015/223; G04G 21/04; A61M 2021/0027; A61M 2021/005; A61M 2205/3553; A61M 2205/3584; A61M 2205/50; A61M 2021/0022

(57) ABSTRACT

The digital command prompting device for dementia patients using augmented reality is an aid is to help all people, especially those who have special needs particularly individuals who have diminished or diminishing function of their brain. The device is predominately mobile but can also be stationary and can be programmed by receiving and selecting pre-set commands to operate and assist a user with their daily living standards or needs but is particularly adapted for use when a user is having a disorientation episode as to place and time. The device also has other various features including an illuminated display panel, a GPS tracking capability, an alarm, an illumination element, solar panel and battery backup components, time and date clocks, is provided water resistance covers and/or material, among other things. The device may be used within the home environment, outdoor environment or a restricted environment, e.g. aged care facility, hospital, pre-school or school.

### CROSS-REFERENCE TO RELATED APPLICATIONS

This application is a continuation-in-part application of co-pending U.S. non-provisional utility patent application Ser. No. 15/256,503 filed Sep. 2, 2016 and U.S. non-provisional utility patent application Ser. No. 15/607,516 filed Mar. 28, 2017, now U.S. Pat. No. 9,922,169.

### FEDERALLY SPONSORED RESEARCH AND DEVELOPMENT

None.

### APPENDICES

None.

### BACKGROUND OF THE INVENTION

Dementia itself is not a disease but is caused by lots of different diseases and the word ‘dementia’ is an umbrella term for the symptoms caused by these diseases such as memory loss, confusion, and personality change. Alzheimer's disease is the most common cause but other dementias include vascular dementia, dementia with Lewy bodies and frontotemporal dementia.

Dementia is not an inevitable part of getting older and although a majority of people with dementia are over 65, the condition is not a normal part of getting older. The likelihood of developing dementia rises with age, but it's not a given that an older person will develop it. In the United Kingdom, for example, over 40,000 people under the age of 65 have dementia and in the United States dementia related diseases are estimated to effect 16.5 million people.

Dementia has a bigger impact on women with more and more women living well into their 80s and half a million women in the United Kingdom are now living with dementia and the condition is the leading cause of death in women in the United Kingdom. In addition, women are also more likely to take on unpaid caring roles for other people with dementia and are more than twice as likely as men to provide intensive, 24-hour care for individuals for dementia related conditions.

Dementia is a global issue even though it is a common myth that dementia is only an issue in the western world. The largest increases in dementia expected over the next 20 years are in places like China, India and Sub-Saharan Africa. Dementia is a truly global health issue, affecting 46.8 million people worldwide.

Dementia is more than just memory loss and although most people associate dementia with memory loss, the condition affects people in a wide variety of ways. For example, these might include changes in behavior, confusion, disorientation, delusions, hallucinations, difficulty communicating, problems judging speeds, distances and even cravings for particular foods. Everyone's experience of dementia is different and there are no treatments to stop the diseases that cause dementia and while some treatments can help people to live with their symptoms a little better, there are no treatments that slow or stop diseases like Alzheimer's. Consequently, this means that the diseases will continue to get worse over time unless new treatments can be found quickly.

A person diagnosed with dementia will have contact with a number of health professionals, usually over a long period of time. This will most likely include the family doctor, medical specialists, therapists, community nurses, and social workers all of whom are potential carers for dementia patients. The degenerative nature of dementia means that families and carers will also have a lot of contact with these health professionals and as the disease in an individual progresses these contacts will almost certainly increase.

Usually the family doctor provides on-going health care, sometimes for both the person with dementia and the family and other carers. This means that the relationship between the doctor, the person with dementia and family and carers is critical. The system and devices of the instant invention, it is believed, will reduce the workload of the family doctor assisting a dementia patient and provide extra help for the carers of these patients.

Today, there are more than 200,000 Australians providing informal unpaid assistance to people with dementia and the vast majority of people with dementia living in the community (91%) rely on an informal carer to support them. Most informal carers are either the spouse or adult child of the person with dementia and nearly a quarter of people with dementia living in the community (22%) rely solely on informal care and do not access any formal care services. Additionally, 81% of co-resident informal carers provide more than 40 hours of care per week for dementia patients. Future projections such as Access Economics projects that by 2029 there will be a shortage of 94,266 full-time equivalent (FTE) family carers. It is further estimated the current cost of replacing family carers with paid carers is $5.5 billion per year.

Therefore, the impact of caring can be enormous, for example, caring for a person with dementia can lead to increased rates of depression, stress and anxiety for carers compared to non-carers. For example, in Australian surveys of carers, 31% of respondents reported that caring for the person with dementia had a negative impact on their physical health (3), and 34% reported feeling weary or lacking in energy. Additionally, the stress of caring may result in impaired immunity, high levels of stress hormones, hypertension (high blood pressure) and an increased risk of cardio-vascular disease for carers.

Additionally, the demands of caring for someone with dementia have been shown to put carers at risk of social isolation. A National Carer Survey in the United States found that carers of people with dementia were more likely to report giving up their holidays or hobbies, having less time for family, having more family conflicts and work related problems.

Additionally, caring for dementia related patients can also have significant financial impacts. According to the Dementia in Australia report, 54% of carers of people with dementia (and 45% of primary carers) are of working age while only 56% of these (and 38% of primary carers) were employed at the time of the survey.

The concept of artificial intelligence, also known as AI, envisions using computers to simulate human intelligence by designing computer programs which mimic certain human analytic functions such a learning and/or problem solving. These computer programs attempt to perceive an environment surrounding a human, in which a human would use their various senses, sight, sound, smell, touch, taste, e.g., and then based on the human's organs and cognitive functions would assess and determine a course of action for solving a problem.

AI requires the application and integration of several different fields including computer science, mathematics, linguistics, and psychology and has seen particular applications in the fields of “robotics” and “machine learning”. By its nature AI is machine based, that is, it uses a machine, a computer, and must use various sensors or systems to analyze the environment, e.g., an optical system to simulate sight, a sound recording system to simulate sound reception, and radar type systems for detecting the location and distances of objects. An optical system could use an object character recognition system for recognizing letters and numbers and also can be used to recognize shapes and forms. Additionally, AI uses various mathematical methods such statistical analysis, search and mathematical optimization, fuzzy logic, etc., to make various AI conclusions.

In the healthcare field, AI can be used to assess a patient or user condition, particularly in high risk cancer patients. AI can also monitor patient conditions, such as with dementia, particularly changes in their cognitive conditions and by matching vital statistical information AI programs can be used to modify patient or user care plans.

There is a need for system and/or devices which may assist patients with Alzheimer's and/or dementia diseases by using long and short-term memory reminders, or prompts, which can enhance the patient(s) memory, and hopefully their cognitive states, especially when they are having psychotic place and time disorientation episodes. Usually, when these episodes occur, medical providers will attempt to determine a patient's mental status by performing a X4 test, which involves asking the patient four questions about who they are, where they are, what time is it, and a most recent event in the patient's life.

It appears that many patient(s) with Alzheimer's and/or dementia diseases have fairly good long-term memory while their short-term memory appears to falter many times causing the patient(s) to have memory loss and/or place and time disorientation episodes. Many times, these episodes can lead to schizophrenia where the patient(s) becomes partially or psychologically disoriented in place and time with associated emotional frustrations. And although this long-term versus short-term memory loss phenomenon is not understood, the fact that their long-term memory is relatively intact provides an opportunity for assisting these patients by creating long-time memory reminders, or prompts, which can be used to a bring them into a present cognitive state of mind. By chronologically ordering the reminders, or prompts, and presenting them to the patient(s), and hopefully triggering those portions of the brain related to long and short-term memory, the system and devices of the invention will gradually orient them as to the present time, place, and/or their situation. The patient(s) reminders, or prompts, make take different forms; however, photos, images and pictures appear to be the most effective, as a type of non-verbal communication, in triggering or causing patient(s) to remember dates, events, or life occurrences re-orienting them into a present cognitive state of mind as to time and place.

The present invention further uses a set of reminders for each patient which are unique to them, in the form of pictures, images, photos, dates, etc., which are loaded and/or encoded into a database for use with the invention system and devices. These reminders are facets and/or periods of the patient(s)' life and life history, both in their work, profession and personal life. The reminders may consist of photos of the patient(s)' family, friends, spouse, sports idols, or other important life individuals close to the patient(s). Additionally, these reminders may include dates, times, and places to assist in orienting the patient as to when and where the photo was taken, or the event occurred. For a sports person, the shape of a ball, racket, hockey stick, etc., could be a reminder. For example, for a farmer, photos and sounds of various livestock, chickens, cows, etc. or equipment such as tractors etc. could be a reminder. For a bookkeeper, accountant, the images of ledgers with figures could be a reminder. Although the reminders and prompts are mostly visual in nature, olfactory or smell sensation, or sounds, could also be used as a reminder; for example, if the patient worked as a gardener, certain plant or flower fragrance could be a unique reminder for that individual. The reminders for a typical patient usually display photos, images, drawings, etc. from his or her early childhood years, school years, working and professional life, marriage life including children and grandchildren. Once a set of reminders are determined for a patient(s), the reminders are screened by a patient(s)' carers, ordered chronologically, loaded and/or encoded into the invention system and devices to be used as reminders or prompts when the patient(s) is having a memory and/or disorientation episode.

### SUMMARY OF THE INVENTION

For the purposes of the following disclosure, the term “patient or patient(s)” denotes a person or persons who has been diagnosed with or identified as having dementia or similar conditions, for example, Alzheimer's disease, and for whom medical care has been prescribed and/or identified while the term “user” denotes a person who has been identified to be a patient and who is using the invention system and/or devices as described in the instant invention; occasionally, in the disclosure, the term “patient/user” is used to denote a “patient” or a “user”. Similarly, the term “carer” refers to any number of health professionals or other individuals involved with providing any time of care, mental, physical, or otherwise for a “user” and/or “patient” and an “administrator” is a person supervising a number of carers.

There exists in the prior art medical alert devices used to assist people, usually elderly or disabled, which devices act to contact or communicate with emergency medical facilities in the event a person has a traumatic medical event. This medical event may be a fall where the person is unable to right themselves, or when they have a stroke or heart attack disabling them from verbally calling for help. These medical alert devices usually consist of a small device attached to a lanyard or other cord necklace to allow the device to be worn by a user and the device usually has a single button to be pushed by the user. The button can then activate a communication component for directing connecting with emergency medical facilities. These medical alert devices however are usually a single function apparatus and usually lack any other capabilities for assisting a user.

These is a need for a system and device which has the capability both to act as a medical alert device but additionally can be used to assist a person through their entire daily and nightly routines by prompting them at the appropriate times to perform certain tasks. There is a particular need for individuals of diminished mental or brain function, either because of being of young age, for example, a preschooler, or for elderly individuals whose mental or brain function is diminishing because of age. The device and system should further be capable of being used by visually or hearing impaired, handicapped or mute individuals The system and device should further be provided with an alarm feature in the form of a prompt, for example, a vibrator or sounder which signals the user to perform a certain task; the device should be able to continue to repeat to vibrate or sound an alarm to get the attention of the user. If after a certain time frame there is no response from the user, the device should switch over to a medical alert signal which is transmitted to a medical alert staff desk, nursing station or emergency office or facility. There is also a need for a system and/or devices which may assist patients with Alzheimer's and/or dementia diseases by using long and short-term memory reminders, or prompts, which can enhance the patient(s) memory, and hopefully their cognitive states, especially when they are having psychotic place and time disorientation episodes.

The instant invention comprises a computer system and digital command prompting devices, to assist and help people with medical needs and assistance, particularly of dementia related conditions, i.e., a user or patient and to assist people with orient them when they are having a location and time disorientation episode.

The system and devices of the instant invention, a digital command prompter with augmented reality, known as the DCP with AR system and devices, fundamentally use digital appliances provided with prompting computer software developed to inform or remind a user to perform, or not to forget to perform, important tasks, events, time slots or memories that make up the unique life cycle of the user. The DCP with AR system and devices also incorporate AI features to enhance the effectiveness of the prompting software particularly for noting or updating changes in a user's conditions. The software can also be used to store memories, update medical information, and link fragmented threads of memory together, of the user, to maintain a level of independence for those living with Alzheimer's and dementia diseases and be used to display a series of personally unique reminders, or prompts, which can be used to re-orient a user or patient when they are having a dis-orientation episode as to time and place.

The DCP with AR further incorporates AI to assist in assessing, analyzing, and ordering reminders and prompts used with the invention. Particularly, the DCP with AR is able to utilize AI for object recognition and tracking gestural input, i.e., non-verbal communication between individuals using various body parts such as hands. For example, gestural recognition and tracking methods allow a medical patient to use their hands and feet/gait to communicate with medical regarding his or her needs or wants.

As discussed above, the DCP with AR is constructed on the basis that each user or patient has a series of unique facets, events, or familiar periods of their life, here known as the DCP with AR life history, whether it was associated to their working history or their personal life. Based on the individual DCP with AR life history, a file is created comprising a series of reminders, usually photos, images, drawings, dates, times, and places, in the form of prompts from the patient's long-term memory are created, and chronologically ordered. When a patient is having a dis-orientation episode the prompts can be presented and displayed to the patient to assist him or her to gradually reorient them to their current date, time and place.

The DCP with AR system and devices are especially constructed for those people who have special medical needs, such as those with dementia, Alzheimer's disease, or similar age-related mental conditions. The DCP with AR computer system is provided with a centralized database constructed to electronically interact, via a wired or wireless electronic communication network, with a centralized computer system. The DCP with AR system and devices are further provided with computer administrative internet web page interfaces, the DCP with AR administration interfaces, and carer internet web page interfaces, the DCP with AR carer interfaces, which allow for the programming of user DCP with AR devices for receiving and selecting pre-set commands to operate and assist a user to help them with all aspects of their daily living. The whole DCP with AR system, and devices, are operated by computer application software, hereinafter “the DCP with AR app”, constructed to operate the DCP with AR computer system and devices. The administrators using the DCP with AR administrative internet web page interfaces are known as DCP with AR administrators and carers using the carer internet web page interfaces are known as DCP with AR carers.

The DCP with AR devices are further constructed to either be mobile or stationary and may come in various applications, particularly in the form of a mobile activator with a lanyard, smartphones, or wearable watches, all provided with the DCP with AR app. Additionally, the DCP with AR watch device is also constructed to be fitted to a wrist bracelet or watch band such that it can be worn in any environment. The DCP with AR app is further constructed to be used with any smartphone or electronic tablet and as with any smartphone or electronic tablet, it can be stationary and sited beside a bed of a user or can also be fitted to be used in an automobile.

Although the DCP with AR system and devices are primarily constructed for a user with special medical needs, as discussed above, it can also be used for a functional but forgetful user and depending on the degree of the diminishing thought process of a forgetful user, which may vary due to their capacity during the day and may cause them to become tired or confused, the commands of the DCP with AR device may increase the efficiency and functionality of these type of individuals.

The DCP with AR system and devices are further capable of being programmed to accommodate any type of physical or mental user disability and they are constructed with daily-calendars as a reminder for the user, e.g., to collect their newspaper or weekly calendar reminder, e.g., on Tuesday: garbage put the bins out on street. The DCP with AR system and devices can also inform the user of activities available in the nursing home or the location of various sections of a hospital, e.g., the x-ray department, a service which the user may need. The DCP with AR system and devices can also be used to remind the user of appointments with doctors which can be pre-set via voice application in DCP with AR device.

The DCP with AR system and devices are further constructed to be used in an aged care facility and can assist the user and facility staff by allowing them to perform his/her own tasks. Similarly, the DCP with AR system and devices can be used as an assessment tool for the user in lieu a personal assessor especially when the user becomes cagey and try to prove they are still 100% okay when the user realizes that they are faltering, mentally or physically, and are attempting to hide this fact from facility staff. The assessment feature/tool can assess a mental or physical competence level of the user for staff doctors or specialists without the awareness of the user. Additionally, the DCP with AR system and devices can assist in crime protection preventing the user from becoming an “easy target” for criminals.

The DCP with AR system and devices are also provided with an alarm component which can be in the form of a sounder and/or vibrator capable of communicating with the user. This alarm component is further capable of alerting the user of instant messages as the mental and/or physical capacity of the user diminishes. The DCP with AR system and devices are further provided with a voice activation component and/or a picture display prompts to be used by a user unable to read. The voice activation component is particularly useful for a visually impaired user.

As previously discussed, the DCP with AR app spans across the DCP with AR computer system and devices prompting users to complete everyday tasks that elude them as the symptoms of Alzheimer's and/or dementia take hold. Additionally, the DCP with AR system and devices are designed to build up a basic platform of health, physical and mental, information about the user while the user takes advantage of the prompting abilities of the DCP with AR app to better cope with their Alzheimer's and/or dementia degrading conditions. As every individual is unique the DCP with AR system and devices will intelligently collate data and behavioral patterns over time to better serve each of its users as well as better assist their carers and administrators in providing care for the user.

Every user has a set of daily tasks that form the structure of their days and weeks to assist them with routine tasks which they may otherwise forget due to Alzheimer's dementia or associated conditions. These tasks will usually have due date/times so that the individuals can keep track of the date, the time of day, what they need to do next and what they need to tick off their list as they complete each of the tasks. For those users who are at a much more advanced level of Alzheimer's or dementia, the tasks will be ticked off by their carer, using the DCP with AR system and devices, to aid in the daily management of the user. This basic yet methodical system keeps track of the users and is used to assess their progress at different intervals of any disease. The aim of the DCP with AR system and devices is to provide a digital record of every user who uses the DCP with AR system and devices and the difficulties that they experience will eventually help medical professionals to better understand the symptoms at every stage of their disease or condition and hopefully increase the chances of eradicating the disease or condition in the future.

With the DCP with AR system and devices, every patient or user is provided with either a DCP with AR device in the form of an electronic tablet, smartphone, and/or wearable watch for the prompting features to work optimally. The sensation or vibration will prompt haptic feedback, and sound alert the user to perform a particular task during the day as often as required or if the patient or user is having a disorientation episode. For example, an alert may alert the patient or user to do a “Who Where When Update”, the user, will then respond a series of life history images, photos, or other unique reminders as prompts which the user or patient must respond. In the event, the reminders or prompts fail to re-orient the patient or user, carer intervention may occur to assist the patient or user.

There are other benefits of the DCP with AR system and devices. For example, if a carer providing center or aged center care have rostered staff taking care of multiple patients daily, they will have the ability to identify which carer signed off on the tasks of any given user/patients. This could ease the transition when onboarding of new carer staff as they will have a diarized set of instructions that they can follow for each user/patient.

### BRIEF DESCRIPTION OF THE DRAWINGS

FIG. 1 discloses a concept flow diagram of the DCP with AR system and devices.

FIG. 2 discloses a first embodiment of the DCP with AR devices 2, 3 and 4.

FIG. 3 discloses details of the DCP with AR devices of FIG. 2 in use.

FIG. 4 discloses a second embodiment of the DCP with AR device.

FIG. 5 discloses details of the DCP with AR device 2 of FIG. 2.

FIG. 6 discloses further details of the DCP with AR device 2 of FIG. 2.

FIG. 7 discloses the DCP with AR devices of FIG. 2 in use.

FIG. 8 discloses details of the DCP with AR device of FIG. 4 in use.

FIG. 9 discloses further details of the DCP with AR device of FIG. 4 in use.

FIG. 10 discloses further details of the DCP with AR device of FIG. 4 in use.

FIG. 11 discloses the DCP with AR device of FIG. 4 in use on a user.

FIG. 12 discloses a flow chart of the operation of the DCP with AR system and devices.

FIG. 13 discloses a questionnaire for use with the DCP with AR system and devices.

FIG. 14 discloses a sample photo for use with the DCP with AR system and devices.

FIG. 15 discloses a second sample photo for use with the DCP with AR system and devices.

FIG. 16 discloses a third sample photo for use with the DCP with AR system and devices.

FIG. 17 discloses a fourth sample photo for use with the DCP with AR system and devices.

FIG. 18 discloses the devices of FIG. 2 of the DCP with AR in use with the photo of FIG. 14.

FIG. 19 discloses the device of FIG. 4 of the DCP with AR in use with the photo of FIG. 14.

### DETAILED DESCRIPTION OF THE INVENTION

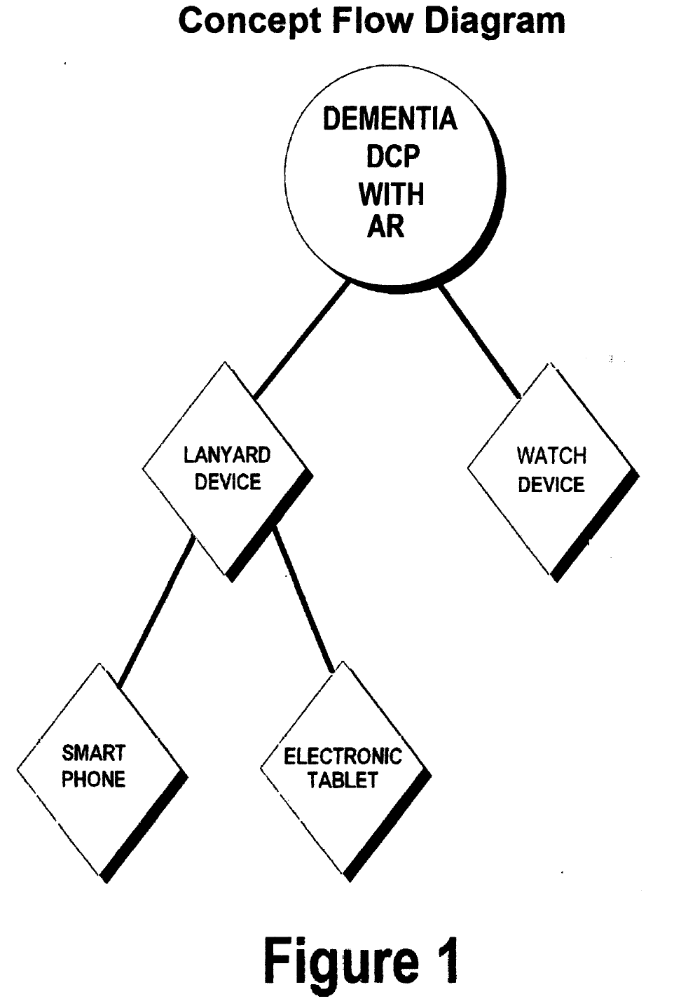

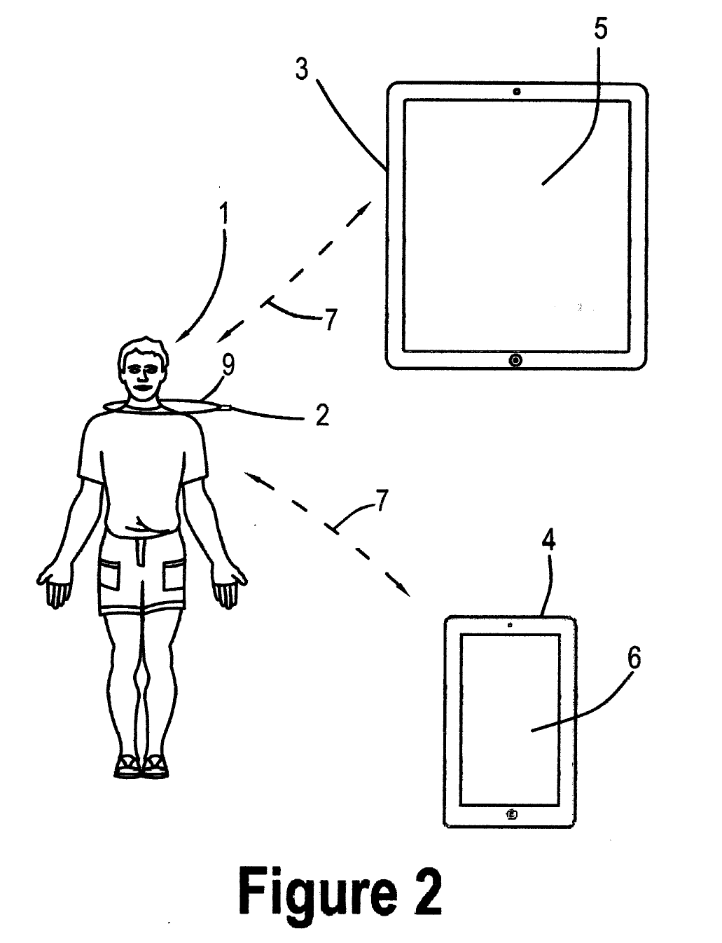

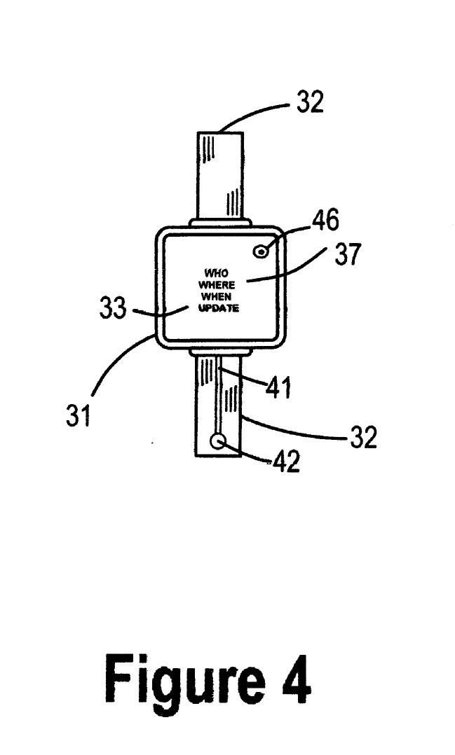

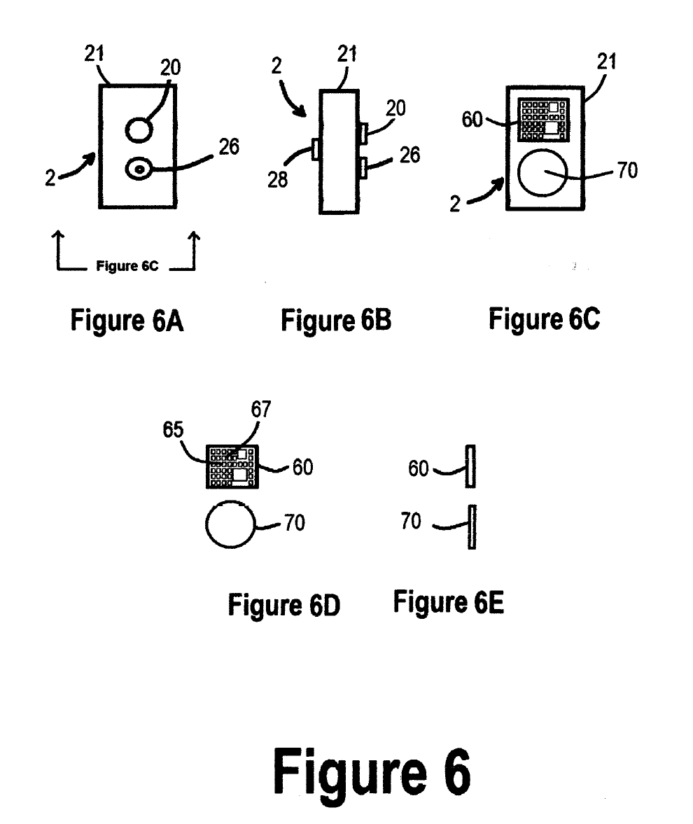

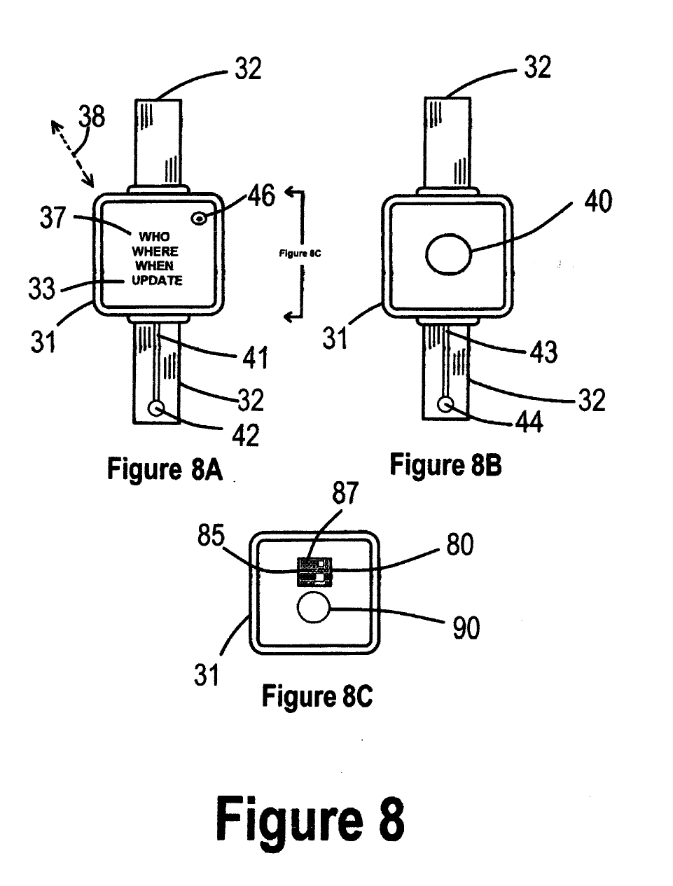

Referring to FIG. 1, a concept flow diagram of the system and devices according to the preferred embodiments of the invention is shown. Particularly in FIG. 1, the overall operation of the preferred embodiments of the invention is disclosed wherein the DCP with AR device, denoted by the empaneled term device may comprise a watch device, e.g., a wrist watchband, bracelet, or a lanyard attachment, etc. As FIG. 1 furthers discloses, the DCP with AR device and system, whether it is either embodiment discussed in reference to FIG. 2 or 4, of the instant invention, is provided with a sensor device capable of creating and transmitting electronic signals, via a wireless signal transmitter imbedded in the sensor device. In the DCP with AR device of FIG. 2, the wireless signals are transmittable to a mobile electronic appliance, such as a smart phone, or an electronic tablet. The mobile electronic appliance is capable of sending, receiving, and processing the wireless signals and/or data and also is compatible with the Android™ mobile operating system, the iPhone™ cellular phone mobile operating system, i.e., iOS™, and other commercially available mobile device operating systems. The DCP with AR device of FIG. 4 has similar capacity of sending, receiving, and processing wireless signals. Additionally, the sensor devices of either DCP with AR devices of FIGS. 2 and 4, use radiofrequency microchip technology, in the form of a microcomputer transmitter processors capable of sending, receiving, processing, and transmitting data and/or signals to and from navigational positioning systems, such as the Global Positioning System for locating the sensor devices which signals and/or data can transmit signals and/or data to the mobile electronic appliance. As is further shown in FIG. 6D, the DCP with AR devices 2, 3 and 4, are provided with or is accessible to computer processing application software, i.e., otherwise known as an app, which app is capable of processing the any signal and/or data receiving by the sensor device. The DCP with AR device of FIG. 4 has a similar app as discussed in reference to FIG. 8C. DCP with AR devices 2 and 31 are further constructed to interact with a system comprising a centralized database constructed to electronically interact, via wired or wireless electronic communication network, with a DCP with AR centralized computer system (not shown) which allow for the programming of user DCP with AR mobile devices for receiving and selecting pre-set commands to operate and assist a patient or user to help them with all aspects of their daily living. The whole DCP with AR system, and DCP with AR devices, are operated by computer application software, hereinafter “the DCP with AR app”, constructed to operate the DCP with AR computer system and devices. The administrators using the DCP with AR administrative internet web page interfaces (not shown) are known as DCP with AR administrators and carers using the carer internet web page interfaces (not shown) are known as DCP with AR carers.

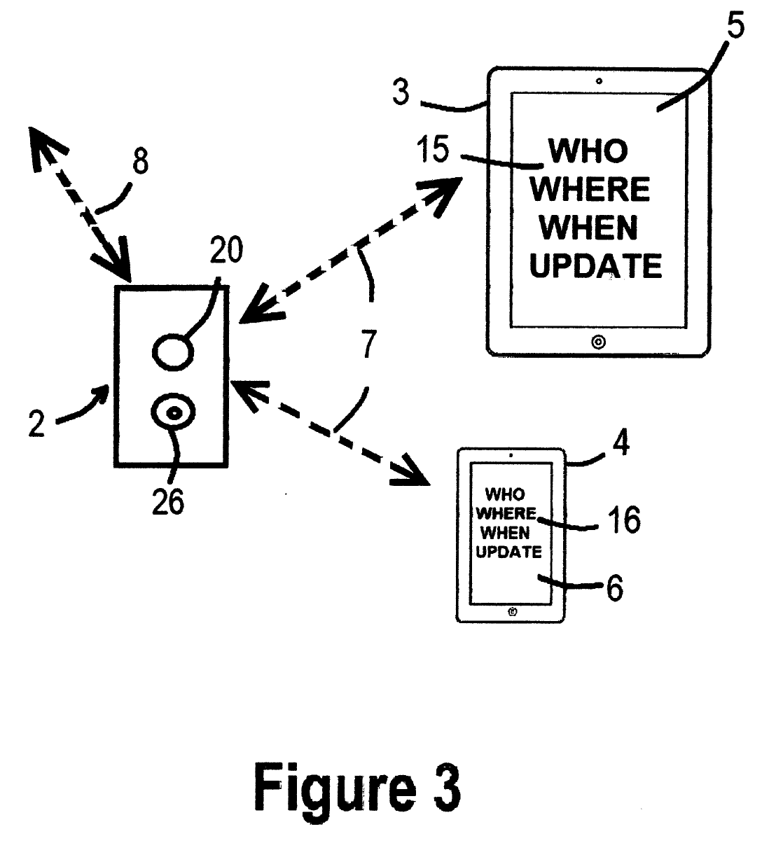

FIG. 2 discloses a first embodiment of the DCP with AR device 2 being utilized by a User 1, in the form of a lanyard or necklace 9. DCP with AR device 2 is shown being attached to lanyard or necklace 9 and is capable of electronically communicating, by wireless signals 7, as is disclosed and discussed below, with an electronic appliance in the form of either an electronic tablet 3 and/or smart phone device 4. Additionally, as is further discussed in FIG. 3, electronic tablet 3 and smart phone device 4 are provided with computer application software 5, and 6, respectively used to assist in operating the electronic tablet and/or smart phone device.

FIG. 3 discloses further the operation of the DCP with AR device 2. Particularly, FIG. 3 shows that DCP with AR device 2 is provided with an activation button 20 which can be depressed by a user; button 20 can be used either for emergencies or can be used to activate a set of commands pre-set by the User. Additionally, the DCP with AR device 2 is capable of creating and transmitting electronic signals, via a wireless signal transmitter imbedded in the DCP with AR device, in the form of a micro-chip transmitter sensor device 60, further disclosed and discussed in reference to FIG. 6. Upon activation, microcomputer sensor transmitter processor 60 sends a wireless signal 7, by various systems, such as a technology that allows electronic devices to a network, i.e., WI-FI, or a wireless technology standard for exchanging data over short distances commonly known as Bluetooth™, or ZigBee™, etc. Additionally, wireless signal 7 can be transmitted to an emergency alert system such medical alert staff desk, nursing station or emergency office or facility. The wireless signal 7 is also transmittable to a mobile electronic appliance, i.e., smart phone 4, or electronic tablet 3. The mobile electronic appliance, either electronic tablet 3 or smart phone 4, is capable of sending, receiving, and processing the wireless signals and/or data and also is compatible with the Android™ mobile operating system, the iPhone™ cellular phone mobile operating system, i.e., iOS™, and other commercially available mobile device operating systems. Additionally, the microcomputer sensor transmitter device processor 60 incorporates radiofrequency microchip technology, discussed further in reference to FIG. 6, capable of sending, receiving, processing, and transmitting data and/or signals 8 to and from navigational positioning systems, such as the Global Positioning System, i.e., GPS, or Global Navigational Satellite System, i.e., the GLONASS. Additionally, microcomputer sensor transmitter device processor 60 is capable, via signal 8, of connecting with the DCP with AR centralized computer system discussed in reference to FIG. 1, supra. Microcomputer transmitter processor 60 is further capable of being coded with information regarding the identity of the User such as his or her birth date, owner, home location, medical records, etc. Additionally, the microcomputer sensor transmitter device 60 is capable of creating a signal and/or data which can be transmitted to a satellite navigational technology system, i.e., GPS or GLONASS, for geographically locating the sensor transmitter device which signals and/or data can transmit signals and/or data to the mobile electronic appliance or smartphone. As is further shown in FIG. 3, the mobile electronic appliance, either electronic tablet 3 or smart phone 4, is provided with or is accessible to computer processing application software, i.e., otherwise known as an “app”, which app is capable of processing any signal and/or data received by the sensor device. For the electronic tablet 3, this app is shown as reference number 5 while for the smart phone 4, the app is shown as reference number 6. Additionally, apps 5 and 6 are capable of producing display or command prompts, displayed respectively as display 15, on electronic tablet 3 and display 16, on smartphone device 4, in the example of FIG. 2, the command “Who Where When Update” to inform, remind or command a user to perform a certain task. Although DCP with AR device 2 is disclosed, as is discussed in reference to FIG. 6, as being powered by a battery, the device is configured to be allowed to be powered with solar energy. Additionally, app 5 or 6 is coded with artificial intelligence techniques for assisting in analyzing data received by DCP with AR device 2.

FIG. 4 discloses a second embodiment of the DCP with AR device. Particularly, FIG. 4 discloses a DCP with AR device 31 housed in the form of watch, having watchband 32, capable of being worn on a hand of a user. Additionally, as FIG. 4 discloses, DCP with AR device 31 has a visual screen display 33, via a computer application program, imbedded in the device, similar to that of DCP with AR device 2 electronic tablet 3 app 5 and/or smart phone 4 app 6, capable of communicating a command message or prompt to the User, here as example “Who Where When Update”. DCP with AR device 31 is configured to be touch activated on its screen although it can be activated by traditional buttons. Additionally, DCP with AR device 31 has an illumination display feature allowing the device to be seen at night or in low-light environments. Further, as is discussed in further detail in reference to FIG. 8, the DCP with AR system is provided with a computer processing application software, i.e., otherwise known as “the DCP with AR app” 85, which app is capable of processing any signal, data, or input from the DCP with AR device 31 and the DC? with AR device app is capable of producing display or command prompts. In the example of FIG. 8, it is the prompt “Who Where When Update”, reference 37 to inform, remind or command the user to update his physical/mental information. Additionally, app 85 is coded with artificial intelligence techniques for assisting in analyzing data received by DC? with AR device 31. Additionally shown in FIG. 4, is a camera feature 46, and sensor 42 and sensor connector 41.

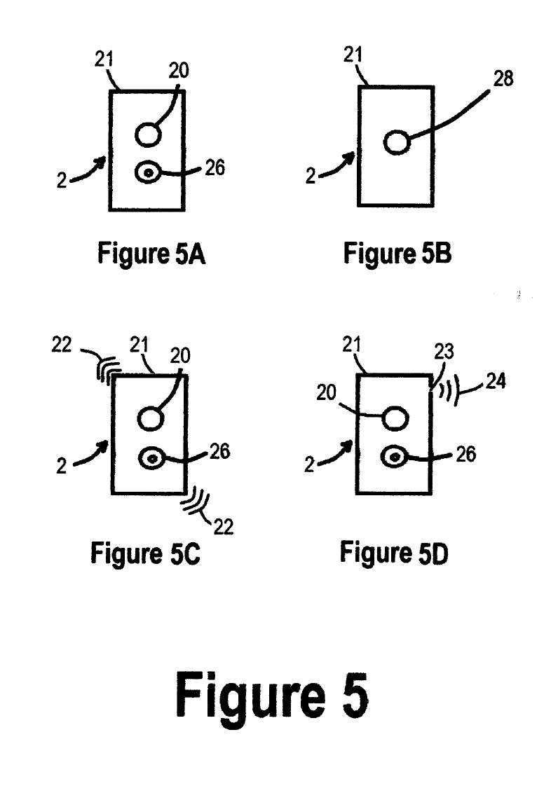

FIG. 5 reveals additional features of the DCP with AR device 2. Particularly, FIG. 5A shows the overall construction of DCP with AR device 2 having a body 21 and activation button 20 and camera 26 while FIG. 5B shows reverse side of device showing sensor 28. FIG. 5C reveals the vibration feature 22 of DCP with A5 device 2 allowing the device to act in an alarm or awakening mode. FIG. 5D reveals a transceiver 23 provided with DCP with AR device 2 capable of producing alarm or awakening sound 24 with transceiver 23 being also capable of receiving voice commands or input.

FIG. 6 is a further detailed figure of DCP with AR device 2 showing details of the device construction. Particularly, FIG. 6A discloses that DCP with AR device 2 has a body 21 and activation button 20 and camera 26 while FIG. 6B is a side view of FIG. 6A showing DCP with AR device 2 in profile with activation button 20, camera 26 and sensor 28. FIG. 6C is a cross-sectional view of FIG. 6A showing internal operational components of DCP with AR device 2, particularly microcomputer sensor transmitter processor 60 and power source 70 in the form of a disc battery. FIG. 6D shows the microcomputer sensor transmitter processor 60 and power source 70 individually revealing their relative size while FIG. 6E is a side profile view of microcomputer sensor transmitter processor 60 and power source 70. As previously discussed, microcomputer sensor transmitter processor 60 is capable of being coded, in app 65, with information regarding the identity of a user and is capable of creating and receiving signals and/or data which can be transmitted to a satellite navigational technology system. Microcomputer sensor transmitter processor 60 also can transmit and receive signals and/or data to the mobile electronic appliance, electronic tablet 3 or smart phone 5. And as previously discussed in reference to FIG. 1, app 65 incorporates artificial intelligence techniques to assist in data received by sensor 28 or inputted thru electronic tablet 3 and smart phone 4.

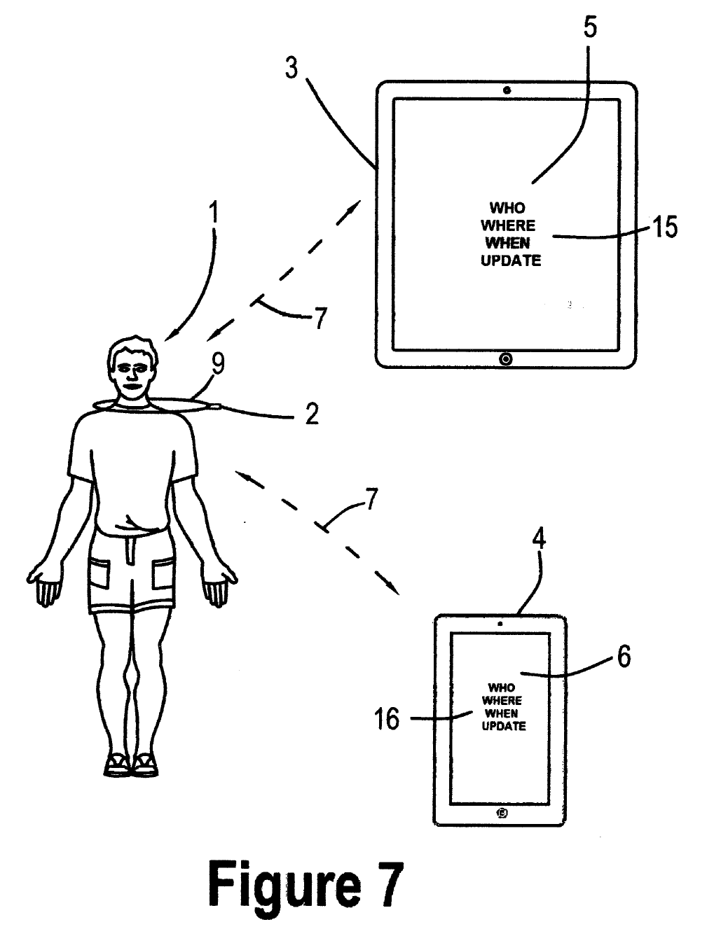

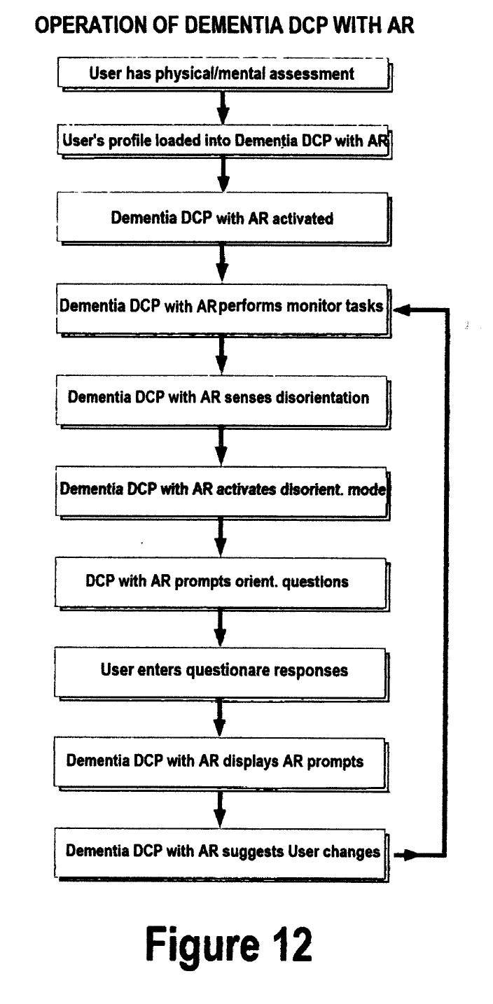

FIG. 7 discloses the DP with AR device of FIG. 2 in use. During its use, a user, or their caretaker, or the DCP with AR may detect or sense a change in the User's condition, particularly of a mental condition or disorientation episode; in that event, the DCP with AR device activates the “Who Where When Update”, i.e., a vital physical statistics analyses mode, to assess potential user condition changes or disorientation status. In this mode, the DCP with AR device prompts the User to respond to a series of questions to assess any mental and/or physical condition changes which may then require the User to alter his/her level of care. The “Who Where When Update” is further discussed in reference to FIG. 12 in an attempt to re-orient the User having a disorientation episode.

FIG. 8 shows details of the DCP with AR device 31 with wristband 32, particularly wireless communication signal feature producing and receiving electronic signals 38. DCP with AR device 31, is internally provided with electronic components similar to microcomputer sensor transmitter processor 60 and power source 70 of DCP with AR device 2. As such, DCP with AR device 31 is capable of electronically communicating with GPS and GLONASS satellite systems for allowing location of wearer of the device, i.e., the User. FIG. 8 shows further details of the DC with AR device 31 of FIG. 4 particularly, FIG. 8A shows DCP with AR device 31 with wristband 32, visual screen 33, visual screen display readout 37, sensor 42 and sensor connector 41. FIG. 8A further reveals the wireless communication signal feature producing and receiving electronic signals 38 from microcomputer sensor transceiver processor 80 of FIG. 8C. FIG. 8B, a rear view of DCP with AR device 31 of FIG. 8A, shows sensor probe 40 and sensor probe 44. Sensor probe 42 is located and positioned on band 32 such that can be held against the chest area of a user to measure the user's respiratory rate while sensor probe 44 is constructed and located on the DCP with AI device 31 to sense the heart rate on the wrist of a user. Sensor probe 44 is further capable of sensing a user's body temperature and is connected to DCP with AR device 31 by connector 43. FIG. 8C, a cross-section view of DCP with AR device 31 of FIG. 8A, reveals that DCP with AR device is internally provided with electronic components 80 and 90 similar to microcomputer sensor transceiver processor 60 and power source 70, respectively, of DCP with AR device 2. As such, DCP with AR device 31 is capable of electronically communicating with GPS and GLONASS satellite systems for allowing location of wearer of the apparatus, i.e., via signal 38, the user and is further capable of connecting with the DCP with AR centralized computer system discussed in reference to FIG. 1, supra. As with DCP with AR device 2, and as discussed supra, in reference to FIG. 4, microcomputer sensor transceiver processor 80 of DCP with AR device 31 incorporates a MEMS™ inertial sensor capable of measuring 1 to 3 axis acceleration, i.e., a pedometer 87, which is processed by imbedded software app 85 capable of calculating calorie levels or other calculations as previously discussed, supra.

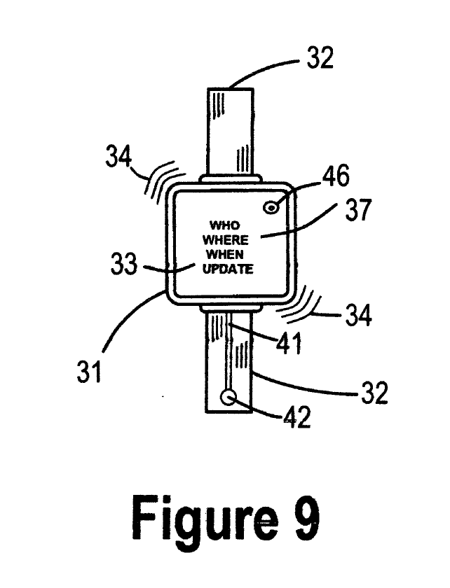

FIG. 9 discloses an additional feature of DCP with AR device 31, particularly, a vibration capability allowing the device to awaken, remind and or alert the User to perform a particular task, here, as an example, to “Who Where When Update” as discussed in reference to FIGS. 7 and 12.

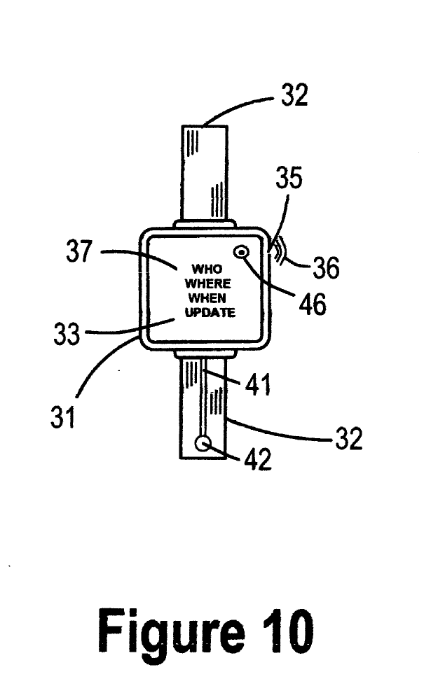

FIG. 10 discloses an additional feature of the DCP with AR device 31, particularly, a sound transceiver 5 capable of emitting sound 36 for alerting the User to awaken, remind him or her to perform a particular task or respond to re-orientation prompts, here, as an example, to “Who Where When Update”. Additionally, transceiver 35 is capable of receiving audible input which can be used to program DCP with AR device 31.

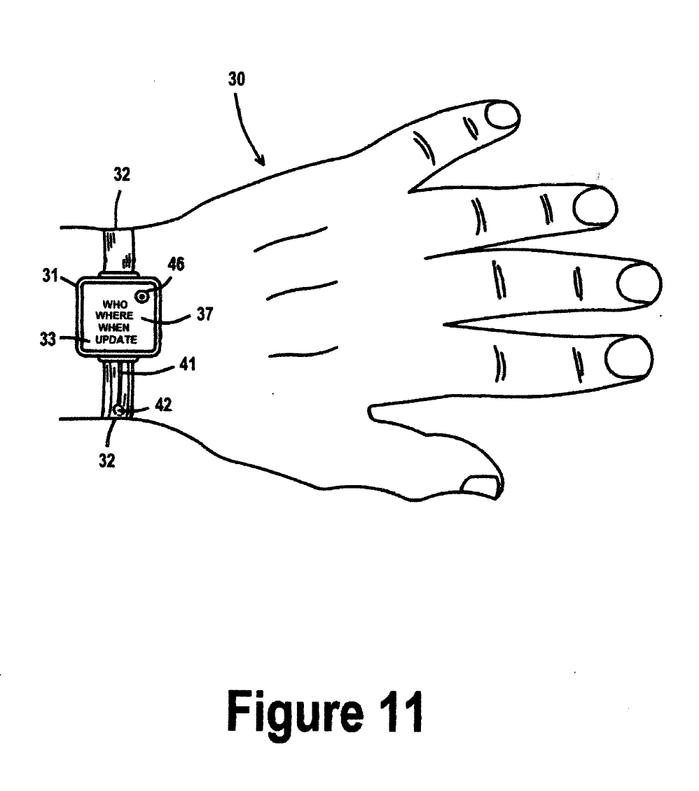

FIG. 11 shows the DCP with AR device 31 of FIG. 7 being utilized by a user, i.e., being worn on the wrist of hand 30 of a user. The DCP with AR device has a visual screen 33 and screen display 37 capable of communicating a readout to the User. The DCP with AR device 31 of FIG. 9 has all of the features and capabilities of the DCP with AR device 31 of FIGS. 5-8. Also seen in FIG. 10, the DCP with AR device 31 is provided with the camera scanner feature 46 along with sensor probe 52 which operates in a similar fashion to sensor probe 42 and sensor probe connector 41 of FIGS. 7-9 and are provided with electronic components 80 and 90 as well as app 85 as discussed in reference to FIG. 4, capable of producing all of the electronic information and processing all of the data of the DCP with AR device 31 of FIGS. 4, 8-10.

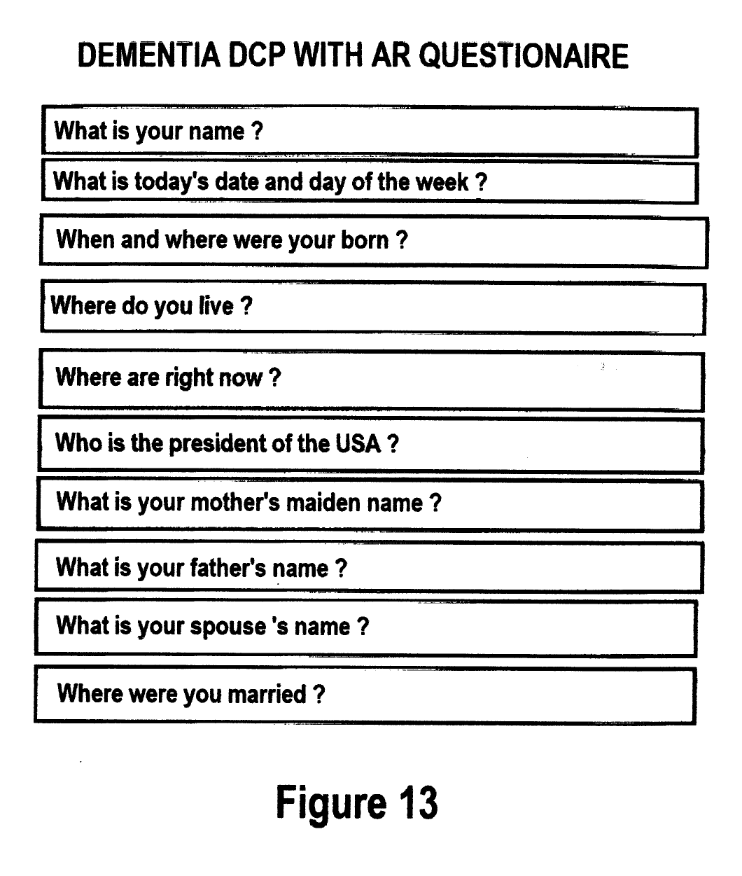

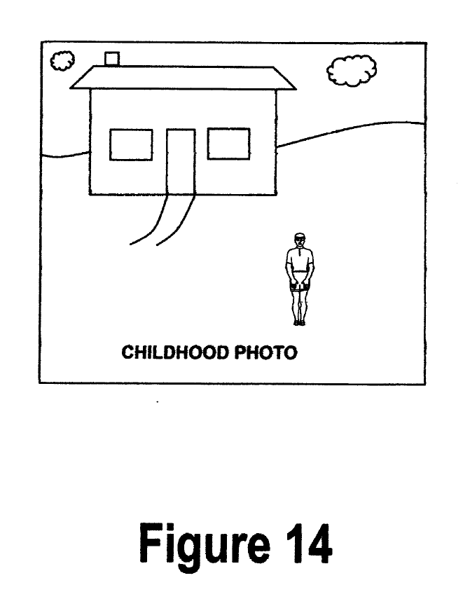

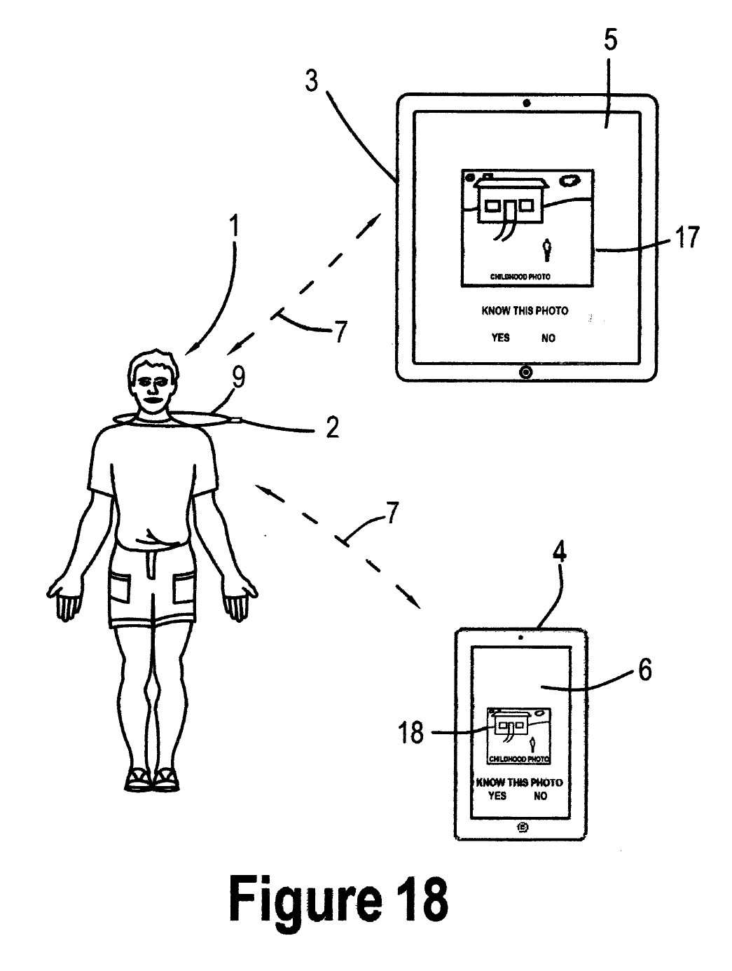

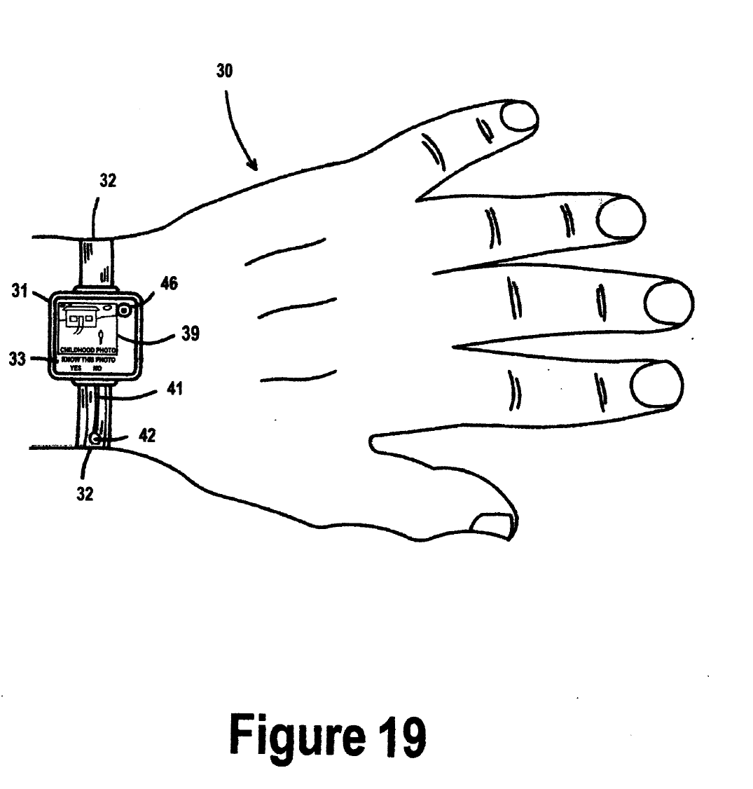

FIG. 12 is a step by step flow chart of an example of various task routines to performed by the DCP with AR device and system in responding to a user dis-orientation episode. Particularly in FIG. 12 is shown that a user undergoes a physical and mental assessment with the results being loaded in the DCP with AR device. Subsequently, the DCP with AR device is activated by the User and during the course of use, the DCP with AR device and system will perform various physical monitoring tasks on the User. In the event, either the User, their caretaker, or the DCP with AR device, detects or senses a disorientation episode of the User, the DCP with AR device activates the “Who Where When Update”, i.e., a vital physical statistics analyses mode, to assess potential User orientation status. The “Who Where When Update” then prompts the User to respond to a series of questions to assess his/her orientation status which responses are entered into the DCP with AR device. Using the User's profile and AI analytical processing techniques, various mathematical methods such statistical analysis, search and mathematical optimization, fuzzy logic, etc. an assessment is made as to the User's orientation. In the event, the DCP with AR device and system concludes the User is disoriented, a series of prompts including photos of the User's profile are displayed, in chronological order in an attempt to orient the User. FIGS. 14 to 17 are samples of photos and/or images which can be used in the orientation serial prompts for the User and depending on the responses by the User, suggestions can be made for further User operation of the DCP with AR device, particularly by the app 65 of DCP with AR device 2 and app 85 of DCP with AR device 31. Particularly, as discussed in reference to FIGS. 18 and 19, when DCP with AR system and devices are in the disorientation mode, a series of prompts in the form of the Patient's photos are displayed with the objective of orienting him or her. Usually, the sequence of the displayed photos proceeds from the long-term memory in progressive chronological order. For example, as shown in FIG. 18 a childhood photo is displayed with a message or query bar, labeled “KNOW THIS PHOTO, YES, NO” below the photo asking whether the User recognizes it; the User then taps on the appropriate response. If the response in “YES”, the series of questions prompted in FIG. 13 are again prompted with the objective of receiving positive responses indicating the User has regained orientation. In the response to the query of FIG. 18 is in the negative, a subsequent profile photo is displayed with a similar query bar being displayed; in the event several of the responses to the query bars are in the negative, a signal and/notice is sent to the User's Carer and/or Administrator to activate an intervention alert of the User.

FIG. 13 is a series of questions which can be used to assess a user's mental state when the DCP with AR is in the “Who Where When Update” mode. For example, the User is queried “What is your name?”, “What is today's date and day of the week?”, “When and where were you born?”, “Where do you live?”, “Where are you right now?”, “Who is the President of the USA?”, “What is your mother's maiden name?”, “What is our father's name?”, “What is your spouse's name?”, “Where were you married?”. These responses are processed using the apps 65 and 85, of DCP devices 2 and 31, respectively incorporating artificial intelligence techniques which then make suggest changes to the User level and type of care and orientation.

FIG. 14 discloses a sample photo or image for use in stimulating a user or patient's long-term memory. Particularly, FIG. 14 displays a childhood photo which includes the User or Patient and his childhood home.

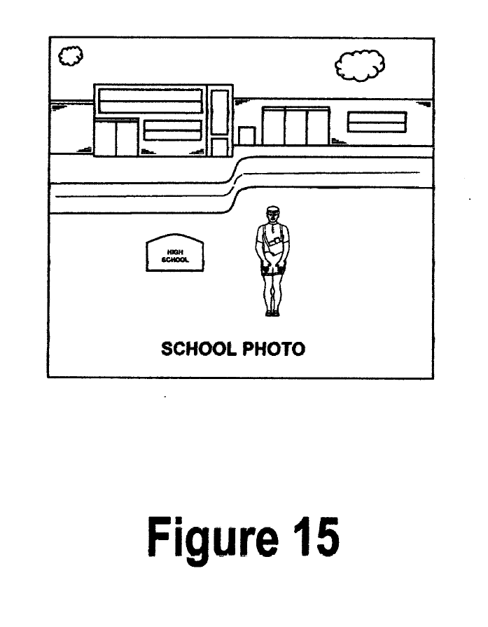

FIG. 15 discloses a second sample photo or image for use in stimulating a user or patient's long-term memory. Particularly, FIG. 15 displays a school photo which includes the User or Patient and his high school.

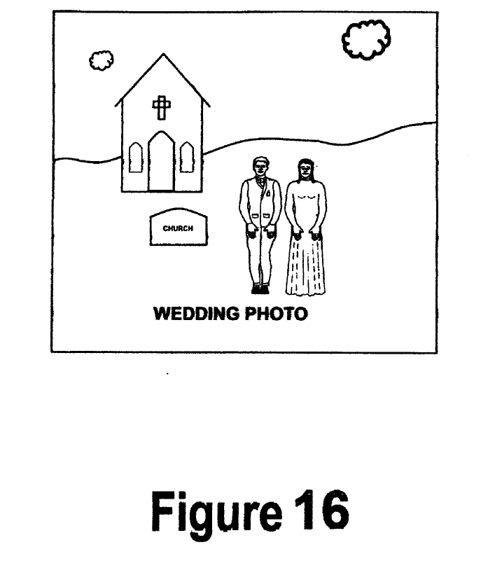

FIG. 16 discloses a third sample photo or image for use in stimulating a user patient's long-term memory. Particularly, FIG. 16 displays a school photo which includes the User or patient on his wedding day in front of his church.

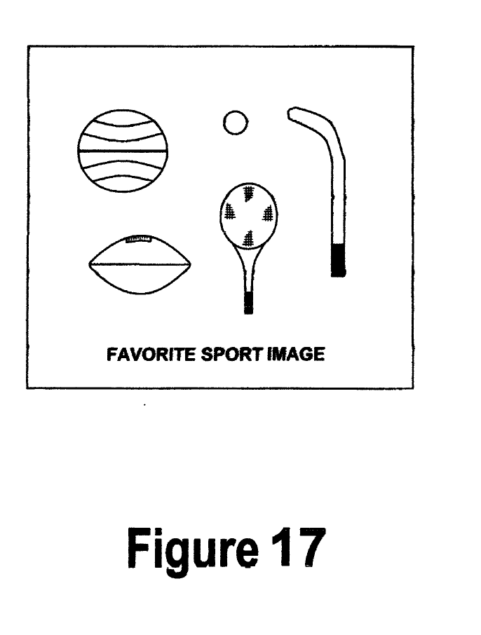

FIG. 17 discloses a third sample photo or image for use in stimulating a user or patient's long-term memory in relation to a profession or hobby involving sports. Particularly, FIG. 17 displays a photo which includes various sports equipment such as a basketball, tennis ball, hockey stick, tennis racket or American football which may be relevant to the User or Patient's long-term memory.

FIG. 18 discloses the devices of FIG. 2 of the DCP with AR in use with the photo of FIG. 14 and its operation. Particularly, FIG. 18 discloses that in the disorientation mode, a series of prompts in the form of the User's photos are displayed with the objective of orienting him or her. Usually, the sequence of the displayed photos proceeds from the long-term memory in progressive chronological order; in FIG. 18 a childhood photo is displayed with a message or query bar, labeled “KNOW THIS PHOTO, YES, NO” below the photo asking whether the User recognizes it; the User then taps on the appropriate response. If the response in “YES”, the series of questions prompted in FIG. 13 are again prompted with the objective of receiving positive responses thereto indicating the User has gained orientation. In the response to the query of FIG. 18 is in the negative, a subsequent profile photo is displayed with a similar query bar being displayed; in the event several of the responses to the query bars are in the negative, a signal and/notice is sent to the User's Carer and/or Administrator to activate an intervention alert of the User.

FIG. 19 discloses the device of FIG. 4 of the DCP with AR in use with the photo of FIG. 14 and operates in the same mode and manner of the devices of FIG. 2 as discussed in reference to FIG. 18.

Additionally, the principles of the invention disclosed herein could be practiced by those of skilled in the art with equivalent alternative constructions. Although the present invention has been described in considerable detail with reference to a certain preferred embodiments thereof, other embodiments are possible. Therefore, the spirit and scope of the appended claims should not be limited to the description of the preferred embodiment(s) contained herein. The invention may be embodied and practiced in other specific forms without departing from the spirit and essential characteristics thereof. The present embodiments are therefore to be considered in all respects as illustrative and not restrictive, the scope of the invention being indicated by the appended claims rather than by the foregoing description; and all variations, substitutions and changes which come within the meaning and range of equivalency of the claims are therefore intended to be embraced therein.

When introducing elements of the present invention or the preferred embodiment(s) thereof, the articles “a”, “an”, “the”, and “said” are intended to mean there are one or more of the elements. The terms “comprising”, “including”, and “having” are intended to be inclusive and mean that there may be additional elements other than the listed elements. Additionally, any description of the exemplary or preferred embodiments is intended to be read in connection with the accompanying drawings, which are to be considered part of the entire written description.

What is claimed:

1: A digital command prompting device using augmented reality for assisting and orienting a user during a disorientation episode comprising
means for electronically communicating said device with an electronic appliance and wherein the device can be activated by the user and further wherein
the device is provided with means which can send and receive electronic signals and further wherein
the device is provided with means to produce a wireless signal which is transmittable to the mobile electronic appliance and further wherein said electronic appliance is provided with a computer processing application software which can process any signal received by the device and further wherein the computer processing application software can produce a visual display on the electronic appliance to inform or command the user to respond to a question for assisting in the orientation of the user during a disorientation episode.

2: The digital command prompting device of claim 1 further provided with means for transmitting to and receiving a wireless signal from a navigational positioning system.

3: The digital command prompting device of claim 2 further provided with means in the form of push button for activating the device by the user.

4: The digital command prompting device of claim 3 further provided with means for causing the device to vibrate.

5: The digital command prompting device of claim 4 further provided with means for causing the device to produce sounds or to receive voice commands or input.

6: The digital command prompting device of claim 5 further provided with a microcomputer sensor transmitter processor.

7: The digital command prompting device of claim 6 further provided with a power source.

8: The digital command prompting device of claim 7 further provided with a lanyard to allow the user to wear the device.

9: The digital command prompting device of claim 2 wherein the device is housed in a watch.

10: The digital command prompting device of claim 9 further provided with means for causing the device to vibrate.

11: The digital command prompting device of claim 10 further provided with means for causing the device to produce sounds or to receive voice commands or input and further wherein the device is provided with means for allowing the device to receive input from the user to assist in the orientation of the user.

12: The digital command prompting device of claim 11 further provided with a watchband.

13: The digital command prompting device of claim 12 further provided with augmented reality in the form of a series of reminders unique to the user for assisting in stimulating the user's memory.

14: The digital command prompting device of claim 1 wherein the appliance comprises an electronic tablet.

15: The digital command prompting device of claim 1 wherein the appliance comprises a smart phone.

16: The digital command prompting device of claim 1 further provided augmented reality in the form of a series of reminders unique to the user for assisting in stimulating the user's memory.

17: The digital command prompting device of claim 1 further provided means for assessing the user's orientation status.

18: The digital command prompting device of claim 1 further provided means for providing a series of questions to be posed to the user in the event of the disorientation of the user.

19: A digital command prompting device using augmented reality for assisting and orienting a user during a disorientation episode, comprising a lanyard or necklace and wherein
the lanyard or necklace is provided with the means which can electronically communicating, by a wireless signal with an electronic appliance in the form of an electronic tablet and/or smart phone and wherein
the device is provided with a button which can be activated by the user and wherein
the device can create and transmit an electronic signal, via a wireless signal transmitter imbedded in the device in the form of a microcomputer transmitter sensor processor and further wherein upon activation the microcomputer sensor transmitter processor sends a wireless signal by either a wireless technology standard for exchanging data over short distances and further wherein the wireless signal can be transmitted to a carer or emergency office or medical facility and further wherein the wireless signal is transmittable to the electronic tablet or smart phone and further wherein the electronic tablet or smart phone can send, receive, and process the wireless signal and is compatible the Android mobile operating system and/or the iPhone™ cellular phone mobile operating system known as and iOS™ and further wherein
the microcomputer sensor transmitter processor can send, receive, process, and transmit datum and/or a signal to and from a navigational positioning system for geographically locating the user and further wherein the electronic tablet or smart phone is provided with or is accessible to computer processing application software which can process a signal datum received by the device and further wherein
the computer processing application software can produce an augmented reality to the user in the form of a visual display consisting of a series of reminders to assist in the orientation of the user and further wherein
the series of reminders are unique to the user for assisting in stimulating the user's memory
the device is powered by a battery and further wherein
the device is provided with means for causing the device to vibrate to act in an alarm or awakening mode and further wherein
the device is provided with means for causing the device to produce a sound or to receive a voice command or input a voice command to assist in orienting the user in the event of a disorientation episode of the user and further wherein
the device is provided with means for allowing the device for assessing the user's orientation status and further wherein
the device is provided means for providing a series of questions to be posed to the user in the event of the disorientation of the user and further wherein
the device is provided with means for allowing the device to receive input from the user to assist in the orientation of the user.

20: A digital command prompting device using augmented reality for assisting and orienting a user during a disorientation episode comprising
a watch housing the device and further provided with a watchband capable of allowing the device to be worn by the user and further
the device can create and transmit an electronic signal via a wireless signal transmitter imbedded in the device in the form of a microcomputer transmitter sensor processor and further wherein upon activation the microcomputer sensor transmitter processor is capable of sending a wireless signal to a carer or emergency office or medical facility and further wherein the device can send, receive, and process the wireless signal and is compatible the Android mobile operating system and/or the iPhone™ cellular phone mobile operating system known as and iOS™ and further wherein
the microcomputer sensor transmitter processor can send, receive, process, and transmit datum and/or a signal to and from a navigational positioning system for geographically locating the user and further wherein the device is provided with or is accessible to computer processing application software which can process a datum signal received by the device and further wherein
the computer processing application software can produce an augmented reality to the user in the form of a visual display consisting of a series of reminders to assist in the orientation of the user and further wherein
the series of reminders are unique to the user for assisting in stimulating the user's memory
the device is powered by a battery and further wherein
the device is provided with means for causing the device to vibrate to act in an alarm or awakening mode and further wherein
the device is provided with means for causing the device to produce a sounds to receive a voice command or input voice command to assist in orienting the user in the event of a disorientation episode of the user and further wherein
the device is provided with means for allowing the device to assess the user's orientation status and further wherein
the device is provided means for providing a series of questions to be posed to the user in the event of the disorientation of the user and further wherein
the device is provided with means for allowing the device to receive input from the user to assist in the orientation of the user.
